(19) United States
(12) Patent Application Publication
(10) Pub. No.: US 2006/0078494 A1
(43) Pub. Date: Apr. 13, 2006

(54) Use of inhibitors of 24-hydroxylase in the treatment of cancer

(75) Inventor: William J. Polvino, Trinton Falls, NJ (US)

Correspondence Address: PALMER & DODGE, LLP;KATHLEEN M. WILLIAMS, 111 HUNTINGTON AVENUE, BOSTON, MA, 02199, US

(73) Assignee: Sapphire Therapeutics, Inc.

(21) Appl. No.: 11/234,552

(22) Filed: Sep. 23, 2005

Related U.S. Application Data
(60) Provisional application No. 60/612,712, filed on Sep. 24, 2004.

(51) Int. Cl. A61K 51/00; A61K 38/09; A61K 31/7072; A61K 31/704; A61K 31/56; A61K 31/4745; A61K 31/59; A61K 31/4196; A61K 31/4172

(52) U.S. Cl. 424/1.11; 514/383; 514/400; 514/341; 514/167; 424/649; 514/15; 514/34; 514/109; 514/49; 514/492; 514/449

(57) ABSTRACT

The present invention relates to a method of treating cancer in a subject. The method comprises administering to a subject suffering from cancer a therapeutically effective amount of a 24-hydroxylase inhibitor in combination with a second amount of a suitable cancer therapeutic. The 24-hydroxylase inhibitor can be coadministered with a chemotherapeutic agent, such an antitumor antibiotic (e.g., mitoxantrone or bleomycin), an alkylating agent (e.g., estramustine or melphalan), a plant alkaloid (e.g., taxenes such as paclitaxel or docetaxel or vinca alkaloids such as vincristine or vinblastine) or a combination thereof. In additional therapy, the 24-hydroxylase inhibitor can be coadministered as an adjuvant to radiation therapy, such as an external beam irradiation or a radioisotope therapy, such as radiopharmaceutical therapy. Further, the 24-hydroxylase inhibitor can be coadministered as part of a combination therapy that includes hormonal ablation.

### RELATED APPLICATIONS

This application claims the benefit of U.S. Provisional Application No.: 60/612,712, filed on Sep. 24, 2004. The entire teachings of that application are incorporated herein by reference.

### BACKGROUND OF THE INVENTION

Cancer is a disease for which many potentially effective treatments are available. However, due to the prevalence of cancers of various types and the serious disease effects, more effective treatments, for example, those with fewer adverse side effects or more successful treatment outcomes, are needed.

Vitamin D is known to play multiple roles. It is best known for its ability to raise the level of plasma calcium by stimulating bone resorption and intestinal calcium absorption. Vitamin D also has been suggested to play a role in the immune system and the reproductive system. Vitamin D has been shown to down regulate the renin-angiotensin system that in turn regulates blood pressure. In addition, vitamin D and its analogs have been shown to inhibit the proliferation of certain cells, for example, certain types of cancer cells.

### SUMMARY OF THE INVENTION

The present invention relates to a method of treating cancer in a subject in need thereof comprising coadministering to said subject suffering from cancer a first amount of a 24-hydroxylase inhibitor and a second amount of a suitable cancer therapeutic, wherein the first and second amounts together comprise a therapeutically effective amount.

In one embodiment, the suitable cancer therapeutic is a chemotherapeutic.

In another embodiment, the suitable cancer therapeutic is radiation therapy.

In yet another embodiment, the suitable cancer therapeutic is hormonal ablation.

In one embodiment, the chemotherapeutic is selected from the group consisting of: paclitaxel, docetaxel, an antitumor antibiotic, an alkylating agent, a plant alkaloid or a combination thereof.

In another embodiment, the radiation therapy is selected from the group consisting of: external beam radiation, radiopharmaceutical agent or a combination thereof.

In yet another embodiment, the hormonal ablation therapy is selected from the group consisting of ovarian ablation, ovarian suppression, tamoxifen, surgical oophorectomy, radiation-induced ovarian failure, medical castration with luteinizing hormone-releasing hormone analogues or a combination thereof.

In a particular embodiment, the 24-hydroxylase inhibitor is administered orally.

In one embodiment, the method of the invention further comprises coadministering calcitriol.

In one embodiment, the cancer is selected from the group consisting of colorectal cancer, esophageal cancer, myelodysplastic syndrome, multiple myeloma, gliomas, non-small cell lung cancer, stomach cancer, acute myeloid leukemia, hepatocellular carcinoma, breast cancer, ovarian cancer or prostate cancer.

In another embodiment, the cancer is colorectal cancer.

In yet another embodiment, the cancer is esophageal cancer.

In one embodiment, the cancer is myelodysplastic syndrome.

In another embodiment, the cancer is multiple myeloma.

In yet another embodiment, the cancer is glioma.

In one embodiment, the cancer is non-small cell lung cancer.

In another embodiment, the cancer is stomach cancer.

In yet another embodiment, the cancer is acute myeloid leukemia.

In one embodiment, the cancer is hepatocellular carcinoma.

In another embodiment, the cancer is breast cancer.

In yet another embodiment, the cancer is ovarian cancer.

In one embodiment, the cancer is prostate cancer.

The inhibitor can be a compound selected from the group consisting of azoles, aminoalkanimidazoles, aminoalkantriazoles, acylated aminoalkanimidazoles, and acylated aminoalkantriazoles. The inhibitor can be an azole compound having a bulky substituent attached at the C-alpha position to the azole. In some embodiments the inhibitor at the C-alpha position is phenyl, naphthyl, thienyl, or pyridyl. The phenyl, naphthyl, thienyl or pyridyl group can be monosubstituted by halogen, (C₁₋₄)alkoxy, (C₁₋₄)alkyl, di-(C₁₋₄)alkylamino or cyano.

In some embodiments the inhibitor is selected from (R)-SDZ-286907, (R)-SDZ-287871, (R)-VAB636, (R)-VID400, and (S)-SDZ-285428. These compounds are depicted in FIG. 1 as compounds Ia, Ib, Ic, Id, and Ie, respectively.

In some embodiments the inhibitor is represented by Formula I 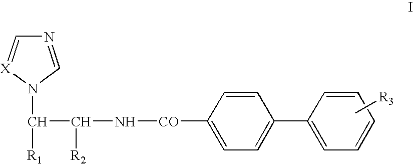
wherein R₁ is phenyl, naphthyl, thienyl or pyridyl; or phenyl, naphthyl, thienyl or pyridyl monosubstituted by halogen, (C₁₋₄)alkoxy, (C₁₋₄)alkyl, di-(C₁₋₄)alkylamino or cyano; and R₂ is hydrogen; or wherein R₁ is hydrogen and R₂ is pyridyl or 2-(5-chloro)pyridyl; and
wherein R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and wherein X is CH or N;
or a pharmaceutically acceptable salt, solvate, or hydrate thereof.

In other embodiments the inhibitor is represented by Formula II 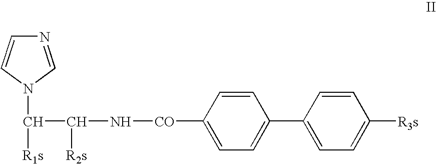
wherein R_{1S} is phenyl, phenyl monosubstituted by halogen, or 1-naphtyl, and R_{2S} is hydrogen; or wherein R_{1S} is hydrogen and R_{2S} is pyridyl or 2-(5-chloro)pyridyl; and wherein R_{3S} is halogen, (C₁₋₄) alkoxy; or a pharmaceutically acceptable salt, solvate, or hydrate thereof.

In certain embodiments the inhibitor is a structural analog of 1,25-(OH)₂ vitamin D₃. For example, the inhibitor may be represented by Formula IV: 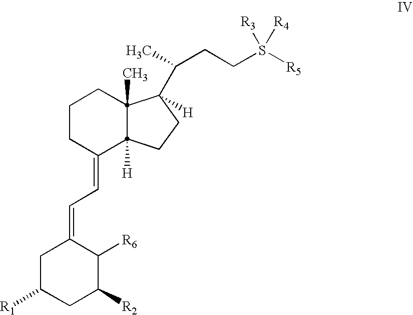
wherein R₁ and R₂ are each independently selected from the group consisting of hydrogen, OR′, —C(O)H, and —C(O)R′;
wherein R′ is selected from the group consisting of a C1 to C₆ alkyl, a cycloalkyl, phenyl, an alkylaryl, an arylalkyl, and a heteroaryl; each of which can be optionally substituted with at least one halogen, thiol, mercapto, hydroxyl, or amino group;
wherein R₃, R₄ and R₅ are each independently selected from the group consisting of hydrogen, hydroxyl, oxy, imine, phenyl, a C1 to C₆ alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R₆ is hydrogen, ═CH₂ or a C₁ to C₆ alkyl, alkenyl, cycloalkyl, or cycloalkenyl, each of which can be optionally substituted with at least one halogen, thiol, mercapto, hydroxyl, or amino group; or a pharmaceutically acceptable salt, hydrate, solvate, ester, or isomer thereof.

Or, the inhibitor may be represented by Formula V: 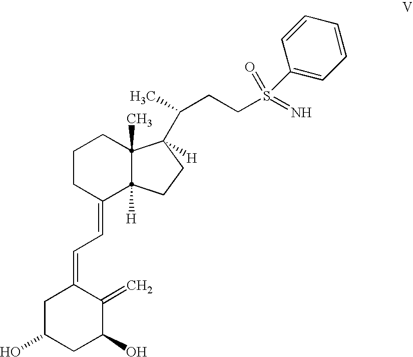
or a pharmaceutically acceptable salt, solvate, hydrate, ester or isomer thereof.

Or, the inhibitor may be represented by Formula VI: 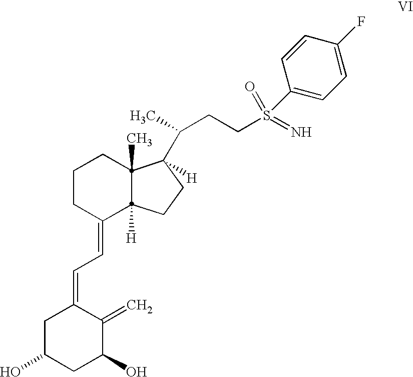
or a pharmaceutically acceptable salt, solvate, hydrate, ester or isomer thereof.

The invention further relates to pharmaceutical compositions useful for the treatment of cancer. The pharmaceutical composition comprises a first amount of a 24-hydroxylase inhibitor and a second amount of a suitable cancer therapeutic. The pharmaceutical compositions of the present invention can optionally contain a pharmaceutically acceptable carrier. The first amount of the 24-hydroxylase inhibitor and the second amount of the suitable cancer therapeutic can together comprise a therapeutically effective amount.

In one embodiment, the cancer treated with a pharmaceutical composition can be selected from the group consisting of colorectal cancer, esophageal cancer, myelodysplastic syndrome, multiple myeloma, gliomas, non-small cell lung cancer, stomach cancer, acute myeloid leukemia, hepatocellular carcinoma, breast cancer, ovarian cancer or prostate cancer.

In another embodiment, the cancer is colorectal cancer.

In yet another embodiment, the cancer is esophageal cancer.

In one embodiment, the cancer is myelodysplastic syndrome.

In another embodiment, the cancer is multiple myeloma.

In yet another embodiment, the cancer is glioma.

In one embodiment, the cancer is non-small cell lung cancer.

In another embodiment, the cancer is stomach cancer.

In yet another embodiment, the cancer is acute myeloid leukemia.

In one embodiment, the cancer is hepatocellular carcinoma.

In another embodiment, the cancer is breast cancer.

In yet another embodiment, the cancer is ovarian cancer.

In one embodiment, the cancer is prostate cancer.

On one embodiment, coadministration of a first amount of a 24-hydroxylase inhibitor and second amount of a suitable cancer therapeutic can result in an enhanced or synergistic therapeutic effect, wherein the combined effect is greater that the additive effect resulting from separate administration of the first amount of the 24-hydroxylase inhibitor and the second amount of the suitable cancer therapeutic.

The invention further relates to use of a pharmaceutical composition comprising a first amount of a 24-hydroxylase inhibitor and a second amount of a suitable cancer therapeutic for the manufacture of a medicament for the treatment of cancer in a subject in need of treatment. The pharmaceutical composition used for the manufacture of a medicament can optionally contain a pharmaceutically acceptable carrier. The first amount of the 24-hydroxylase and the second amount of the suitable cancer therapeutic can together comprise a therapeutically effective amount.

### BRIEF DESCRIPTION OF THE DRAWINGS

FIGS. 1A and 1B depict the structures of selected azole-type 24-hydroxylase inhibitors.

FIGS. 2A and 2B depict the structures of selected analogs of 1,25-(OH)₂ vitamin D₃ which are useful as 24-hydroxylase inhibitors.

FIG. 3 shows growth inhibitory effects of (S)-SDZ-285428 on the prostate cancer cell line PC-3 when added to serial dilutions of a fixed ratio of calcitriol:docetaxel. See Example 1 for details.

FIG. 4 shows an isobologram analysis of the effects of (S)-SDZ-285428 in addition to calcitriol and docetaxel. See Example 1 for details.

### DETAILED DESCRIPTION OF THE INVENTION

The present invention relates to a method of treating cancer in a subject in need thereof comprising coadministering to said subject suffering from cancer a first amount of a 24-hydroxylase inhibitor and a second amount of a suitable cancer therapeutic, wherein the first and second amounts together comprise a therapeutically effective amount.

As defined herein, cancer refers to tumors, neoplasms, carcinomas, sarcomas, leukemias, lymphomas and the like. Suitable cancers include, but are not limited to, colorectal cancer, esophageal cancer, myelodysplastic syndromes, multiple myeloma, gliomas, non-small cell lung cancer, stomach cancer, acute myeloid leukemia, hepatocellular carcinoma, breast cancer, ovarian cancer and prostate cancer.

As used herein, a suitable cancer therapeutic refers to chemotherapeutic therapy, radiation therapy, hormonal ablation therapy or a combination thereof.

### COLORECTAL CANCER

Cancer of the large intestine and rectum (colorectal cancer) is the second most common type of cancer and the second leading cause of cancer death in Western countries. It develops as the result of a pathologic transformation of normal colon epithelium to an invasive cancer. While surgery is most often performed to remove polyps and tumors, chemotherapeutic methods are also employed for the treatment of colorectal cancer. 5-fluorouracil is the most commonly administered chemotherapeutic agent for the treatment of colorectal cancer, and is typically administered by continuous or 48 hour infusion. Two newer drugs, capecitabine (Xeloda) and tegafur with uracil (Uftoral) are very similar to 5FU but which can be taken as tablets, convert to 5-fluorouracil upon internalization. In addition, oxaliplatin and irinotecan have emerged as alternatives, providing increased therapeutic choice in the first-line setting and effective salvage therapies.

### ESOPHAGEAL CANCER

The esophagus is a muscular tube that connects the mouth to the stomach and carries food to the stomach. There are two main types of esophageal cancer: squamous cell carcinoma and adenocarcinoma. At one time, squamous cell carcinoma was by far the more common of the two cancers and was responsible for almost 90% of all esophageal cancers. However, more recent medical studies show that squamous cell cancers make up less than 50% of esophageal cancers today. Still, squamous cell carcinoma remains one of the most common neoplasms in the world, affecting approximately 350,000 people annually worldwide (Parkin et al., (1993) Int. J. Cancer 54: 594-606). Tobacco and alcohol are two major etiological factors in oral cavity squamous cell carcinoma (Binnie et al. (1983) J. Oral Pathol., 12: 11-29).

The success of chemotherapy as a single modality is limited, yet it remains one of the most common treatment for cancer of the esophagus. Other common treatment modalities include surgery to remove part of the esophagus and nearby lymph nodes (esophagectomy), chemotherapy or radiation therapy, or combinations of therapies. Radiation therapy is successful in relieving dysphagia in approximately 50% of patients. Combined therapeutic methods (i.e., chemotherapy plus surgery, or chemotherapy and radiation therapy plus surgery) are under clinical evaluation. Common chemotherapeutic agents for the treatment of esophageal cancer include cisplatin and 5-fluorouracil, admininstered, for example, intravenously by continuous infusion for 4 days.

### MYELODYSPLASTIC SYNDROMES

Myelodysplastic syndromes (MDS) are a heterogeneous group of conditions caused by abnormal blood-forming cells of the bone marrow. In MDS the bone marrow cannot produce blood cells effectively, and many of the blood cells formed are defective. These abnormal blood cells are usually destroyed before they leave the bone marrow or shortly after entering the bloodstream. As a result, patients have shortages of blood cells, which are reflected in their low blood counts. About twenty percent of cases arise in patients who have received either chemotherapy or radiotherapy as part of their treatment for another disease.

Although MDS has not been considered cancer in the past, most hematologists (specialists in diseases of the blood) now consider it is a form of cancer. The major reason is that it is considered a clonal disease with a single population of abnormal cells. That means that all the cells are exactly alike. This is often seen in cancer where all the cells have started from an original abnormal cell. A second reason is that in about 30% of MDS cases, the abnormal bone marrow cells eventually progress into acute leukemia, a rapidly growing cancer of bone marrow cells. Some doctors think MDS is an early form of leukemia although it may never progress into leukemia.

A number of therapeutic agents are currently being tested for the treatment of MDS with some success, for example, anti-angiogenic agents such as thalidomide (Raza et al. (2001). Blood. 2001; 98:958-965; Moreno-Aspitia et al. (2002) Blood 100:96); lenalidomide (CC-5013) (List et al., (2002) Blood 100:139. List et al. (2005) N Engl J Med, 352:549-57); receptor tyrosine kinase inhibitors such as SU5146 (Giles et al (2003) Blood 102:795-801; Albitar et al., (2001) Blood 2001; 98:110); arsenic trioxide (Trisenox®, Cell Therapeutics Inc., Seattle, Wash.) (List et al. (2003) Blood 102:423; Raza et al., (2002) Blood 100:795) and bevacizumab (Avastin®, Genentech, S. San Francisco, Calif.) (Gotlib et al., (2003) Blood 102 (Suppl):425a); as well as famesyltransferase inhibitors, such as tipifarnib (R115777, Zamestra®; Janssen Pharmaceuticals, Beerse, Belgium, and Spring House, Pa.) and lonafamib (SCH66336 or Sarasar®; Schering-Plough Research Institute, Kenilworth, N.J.) (Kurzrock et al. (2003) Blood. 102:4527-4534; Kurzrock et al. (2004) J Clin Oncol. 22:1287-1292.; Feldman et al. (2003) Blood 102(Suppl):421).

### MULTIPLE MYELOMA

Multiple myeloma is a type of cancer formed by cancerous plasma cells in the blood. Normal plasma cells are an important part of the body's immune system.

When plasma cells grow out of control, they can form a tumor called myeloma. Myeloma tumors can grow in many places, including bone marrow. Tumors that grow in more than one place are called multiple myeloma. The myeloma cells interfere with the functions of the bone marrow to make red blood cells, platelets, and white blood cells. According to the International Myeloma Foundation, there are over 13,500 new cases of myeloma in the U.S. each year, representing twenty percent of blood cancers, and one percent of all types of cancer. Mustards like melphalan and other chemotherapeutic drugs such as doxorubicin, cyclophosphamide and vincristine are commonly administered, often in combination with corticosteroids. Thalidomide has been administered to patients whose multiple myeloma is worsening with other treatments. Strong analgesics and radiation therapy directed at the affected bones can help relieve bone pain, which can be severe.

### GLIOMAS

Gliomas are primary brain tumors which arise from the glial cells in the brain and spinal cord, and are the most common primary brain tumors. Gliomas are classified into several groups based on the type of glial cell involved. For example, astrocytomas, which are the most common type of gliomas, are developed from astrocytes. Types of astrocytomas include well-differentiated, anaplastic, and glioblastoma multiforme. Other types of glioma include ependymomas, oligodendrogliomas, ganglioneuromas, mixed gliomas, brain stem gliomas, optic nerve gliomas, meningiomas, pineal tumors, pituitary adenomas, and primitive neuroectodermal tumors, such as medulloblastomas, neuroblastomas, pineoblastomas, medulloepitheliomas, ependymoblastomas and polar spongioblastomas.

Despite extensive treatment efforts, the prognosis for patients suffering from malignant glioma is poor. The search for new treatment modalities as well as improving the efficacy of conventional chemotherapy and radiotherapy is therefore of utmost importance. Malignant glioma is morphologically characterised by extensive pathological neovascularisation, and microvascular density (MVD) is a negative prognostic marker in both low-grade and high-grade glioma (Leon et al (1996) Cancer 77, 362-72; Abdulrauf et al, (1998) J Neurosurg. 88, 513-20). The neovascularisation is controlled by several different growth regulatory factors, of which vascular endothelial growth factor (VEGF) is one of the most important.

Chemotherapy agents used in the treatment of glioma include temozolomide and methotrexate.

### NON-SMALL CELL LUNG CANCER

Non-small cell lung cancer (NSCLC) is the most common type of lung cancer, and is a heterogeneous aggregate of at least 3 distinct histologies of lung cancer including epidermoid or squamous carcinoma, adenocarcinoma, and large cell carcinoma.

Non-small cell lung cancer is less receptive to chemotherapeutic treatment. Standard treatment of small tumors is surgery and radiotherapy; where the tumor has spread within the chest area, radiotherapy is the standard treatment. Chemotherapy is given after such standard treatments, and agents such as cisplatin have been found to be effective in helping patients live longer. In addition, adjuvant chemotherapy, for example, with at least 240 mg of cisplatin and 80 mg of cyclophosphamide, respectively per square meter of body-surface area, improves survival among patients with completely resected non-small-cell lung cancer (Arriagada et al. (2004) N Engl J Med., 350:351-60).

### STOMACH CANCER

Stomach cancer is the second most common human malignancy in the world. About 99% of stomach cancers are adenocarcinomas. Other stomach cancers are leiomyosarcomas (cancers of the smooth muscle) and lymphomas. While the exact causes are not yet understood, a number of causes and risk factors have been associated with an increased risk of stomach cancer, including: Helicobacter pylori (H. pylori) infection, pernicious anaemia, a diet high in salt and foods that are smoked or cured, family history, type A blood group, smoking, and atrophic gastritis.

To treat stomach cancer, surgery is often performed to remove the affected tissue. Chemotherapy or radiotherapy also may be used, especially before surgery to try to reduce a large tumor so it can be operated on, or after surgery to prevent the cancer from coming back (adjuvant therapy). Chemotherapy or radiotherapy may also be used to slow and manage symptoms in advanced stages of the cancer. Drugs most commonly used to treat stomach cancer include the combination ECF, composed of a mixture of epirubicin, cisplatin, and 5-fluorouracil. Other combinations commonly employed include FAMTX (doxorubicin, and methotrexate) and FEMTX (5FU, epirubicin and methotrexate).

### ACUTE MYELOID LEUKEMIA

Acute myeloid (myelocytic, myelogenous, myeloblastic, myelomonocytic) leukemia is a life-threatening disease in which myelocytes (the cells that normally develop into granulocytes) become cancerous and rapidly replace normal cells in the bone marrow. The leukemic cells accumulate in the bone marrow and destroy and replace cells that form normal blood cells. They are released into the bloodstream and transported to the other organs where they continue to grow and divide.

Treatment of AML usually involves a combination of chemotherapeutic agents, for example, cytarabine (ara-C) and an anthracycline drug such as daunorubicin or idarubicin. A third drug, 6-thioguanine, is sometimes added. Granulocyte colony stimulating factors (filgrastin, sargramostim) may also be administered to improve white blood cell counts, and may improve the response to the chemotherapy. This intensive therapy, which usually takes place in the hospital, typically lasts one week.

### HEPATOCELLULAR CARCINOMA

Hepatocellular carcinoma (HCC) is a cancer that begins in the liver cells. HCCs are the most common type of cancer originating in the liver (primary liver cancer), and is one of the leading malignancies worldwide, especially prevalent in the Asia and Pacific regions. More than 1 million people develop into HCC each year (Bosch & Munoz. Epidemiology of hepatocellular carcinoma. In Bannsch & Keppler, eds. Liver cell carcinoma. Dordrecht: Kluwer Academic, 1989; 3-12). The five year survival rate of HCC is quite low (less than 5%). A number of etiological factors, particularly hepatitis B virus (HBV) infection, are involved in the occurrence and progression of HCC.

Chemotherapeutic agents which have been used with some success for HCC include doxorubicin, 5-fluorouracil, and gemcitabine.

### BREAST CANCER

Breast cancer is classified by the kind of tissue in which it starts and by the extent of its spread. Breast cancer may start in the milk glands, milk ducts, fatty tissue, or connective tissue. Different types of breast cancers progress differently. Generalizations about particular types are based on similarities in how they are discovered, how they progress, and how they are treated. Some grow very slowly and spread to other parts of the body (metastasize) only after they become very large. Others are more aggressive, growing and spreading quickly. However, the same type of cancer may progress differently in different women.

In situ carcinoma, which means cancer in place, is an early cancer that has not invaded or spread beyond its point of origin. In situ carcinoma accounts for more than 15 percent of all breast cancers diagnosed in the United States.

About 90 percent of all breast cancers start in the milk ducts or milk glands. Ductal carcinoma in situ starts in the walls of milk ducts. It can develop before or after menopause. This type of cancer occasionally can be felt as a lump and may appear as tiny specks of calcium deposits (microcalcifications) on mammograms. Ductal carcinoma in situ is often detected by mammography before it is large enough to be felt. It is usually confined to a specific area of the and can be totally removed by surgery. If only the ductal carcinoma in situ is removed, about 25 to 35 percent of women develop invasive cancer, usually in the same breast.

Lobular carcinoma in situ, which starts in the milk glands, usually develops before menopause. This type of breast cancer, which cannot be felt or seen on mammograms, is usually found incidentally on mammography during investigation of a lump or other abnormality that is not lobular carcinoma in situ. Between 25 and 35 percent of women who have it develop invasive breast cancer eventually—sometimes after as long as 40 years—in the same or opposite breast or in both breasts.

Invasive breast cancers, which can spread to and destroy other tissues, may be localized (confined to the breast) or metastatic (spread to other parts of the body). About 80 percent of invasive breast cancers are ductal and about 10 percent are lobular. The prognosis for ductal and lobular invasive cancers is similar. Other less common types of cancer, such as medullary carcinoma and tubular carcinoma (which start in milk glands), have a somewhat better prognosis.

A number of treatment options exists for breast cancer, including surgery, radiation therapy, hormone therapy, chemotherapy, and biological therapy, or combinations of these therapies (e.g., mastectomy combined with radiation therapy and/or chemotherapy). Radiation therapy for treatment of breast cancer can be performed using external radiation or by brachytherapy. Standard radiation therapy following a lumpectomy consists of a limited dose of radiation (50 Gy) to the entire affected breast. Biological therapy can be performed using anti-estrogens, for example, using selective estrogen-receptor modulators (SERMs; e.g., tamoxifen, raloxifene) which have been shown inhibit the effects of estrogen on breast cancer cells. Tamoxifen (Nolvadex®) is usually taken for up to five years after mastectomy to prevent recurrence. Fulvestrant (Faslodex®) acts by destroying estrogen receptors in breast cancer cells, and is used to treat metastatic breast cancer in postmenopausal women who did not respond to tamoxifen therapy. Goserelin (Zoladex®), a synthetic luteinizing hormone-releasing hormone (LHRH) is also used to treat metastatic breast cancer in premenopausal women. This medication signals the body to stop producing estrogen, depriving the tumor of the estrogen it needs to grow. Aromatase inhibitors (e.g., anastozole [Arimidex®], letrozole [Femara®], exemestane [Aromasin®]) inhibit the action of the enzyme aromatase, thereby interfering with estrogen production in postmenopausal women.

Among chemotherapeutic agents, the combination most commonly prescribed to treat breast cancer is doxorubicin (Doxil®) and cyclophosphamide (Cytoxan®). Paclitaxel is often prescribed after this combination treatment, if breast cancer has metastasized to the lymph nodes. It is also prescribed following breast cancer surgery. Other chemotherapy drugs include docetaxel and gemcitabine. Biological therapy (e.g., immunotherapy) involves Herceptin® (trastuzumab) to inhibit tumor growth. It also may be combined with chemotherapy as a first line treatment for metastatic breast cancer and may be used after chemotherapy or anti-estrogen therapy to improve the effectiveness of the treatment.

### OVARIAN CANCER

Ovarian cancer is cancer that begins in the cells that constitute the ovaries, including surface epithelial cells, germ cells, and the sex cord-stromal cells. Almost 70 percent of women with the common epithelial ovarian cancer are not diagnosed until the disease is advanced in stage—i.e., has spread to the upper abdomen (stage III) or beyond (stage IV). The 5-year survival rate for these women is only 15 to 20 percent, whereas the 5-year survival rate for stage I disease patients approaches 90 percent and for stage II disease patients approaches 70 percent.

There are many types of tumors that can start in the ovaries. Some are benign, or noncancerous, and the patient can be cured by surgically removing one ovary or the part of the ovary containing the tumor. Some are malignant or cancerous. The treatment options and the outcome for the patient depend on the type of ovarian cancer and how far it has spread before it is diagnosed.

Ovarian tumors are named according to the type of cells the tumor started from and whether the tumor is benign or cancerous. The three main types of ovarian tumors are epithelial tumors, germ cell tumors and stromal tumors.

Epithelial ovarian tumors develop from the cells that cover the outer surface of the ovary. Most epithelial ovarian tumors are benign. There are several types of benign epithelial tumors, including serous adenomas, mucinous adenomas, and Brenner tumors. Cancerous epithelial tumors are carcinomas. These are the most common and most deadly of all types of ovarian cancers. There are some ovarian epithelial tumors whose appearance under the microscope does not clearly identify them as cancerous; these are called borderline tumors or tumors of low malignant potential (LMP tumors). Epithelial ovarian carcinomas (EOC's) account for 85 to 90 percent of all cancers of the ovaries.

Ovarian germ cell tumors develop from the cells that produce the ova or eggs. Most germ cell tumors are benign, although some are cancerous and may be life threatening. The most common germ cell malignancies are maturing teratomas, dysgerminomas, and endodermal sinus tumors. Germ cell malignancies occur most often in teenagers and women in their twenties.

Ovarian stromal tumors develop from connective tissue cells that hold the ovary together and those that produce the female hormones, estrogen and progesterone. The most common types among this rare class of ovarian tumors are granulosa-theca tumors and Sertoli-Leydig cell tumors. These tumors are quite rare and are usually considered low-grade cancers, with approximately 70 percent presenting as stage I disease.

The following drugs are the most common “first-line” treatment options for ovarian cancer: Platinol® (Cisplatin); Paraplatin® (carboplatin); Taxol® (paclitaxel); Alkeran® (melphalan); Adriamycin® or Rubex® (doxorubicin). Two drugs—hexamethylamine (Hexalen®, altretamine) and topotecan hydrochloride (Hycamtin®)—have been approved by the Food and Drug Administration (FDA) for use as second-line, or “salvage,” agents in ovarian cancer (for example, metastatic ovarian cancer patients in whom initial or subsequent chemotherapy with paclitaxel and cisplatin has failed).

### PROSTATE CANCER

Prostate cancer is the most commonly diagnosed cancer in men in the United States and is the second leading cause of cancer-related death in men following lung cancer. There are approximately 200,000 new cases of prostate cancer diagnosed annually and approximately 30-40,000 deaths annually from prostate cancer in the U.S.

While cancer of the prostate is extremely common, its exact cause is not known. When prostatic tissue is examined under a microscope either after prostate surgery or at autopsy, cancer is found in 50 percent of men over age 70 and in virtually all men over age 90. Most of these cancers never cause symptoms because they spread very slowly; however, some prostate cancers do grow more aggressively and spread throughout the body. Although fewer than three percent of the men with the disease die of it, prostate cancer is still the second most common cause of cancer death in men.

A wide array of treatments for prostate cancer have been developed including surgery (e.g., radical prostatectomy), radiation (e.g., external beam radiation Therapy (EBRT), three-dimensional conformal radiation therapy (3DCRT), intensity modulated radiation therapy (IMRT), conformal proton beam radiation therapy, brachytherapy etc.), hormone therapy (including LHRH agonists, oral estrogen drugs, anti-androgens orchiectomy, etc.), chemotherapy (drugs approved by the FDA include: Taxotere® (docetaxel), Novantrone® (mitoxantrone hydrochloride) and Emcyt® (estramustine sodium phosphate)), dietary changes and the use of various herbal supplements.

### EXPERIMENTAL ANIMAL MODELS

Colon adenocarcinoma in rodents induced by the procarcinogen 1,2-dimethylhydrazine and its metabolite azoxymethane (AOM) is a well-characterized carcinogen-induced tumor because of its morphological similarity to human colon cancer, high reproducibility and relatively short latency period (Shamsuddin, (1986) Human Path. 17:451-453; herein incorporated by reference). This rodent tumor model is similar to human colon adenocarcinoma not only in its morphology (Shamsuddin & Trump, (1981) J. Natl. Cancer Inst. 66:389-401) but also in the genes that are involved in tumorigenesis (Shivapurkar et al., (1995) Cancer Lett. 96:63-70; Takahashi et al., (2000) Carcinogenesis 21:1117-1120).

In addition to chemical carcinogen-induced models of colon cancer in rodents, gene disruption of the catalytic subunits of phosphoinositide-3-OH kinase (PI3-Kγ) (Sasaki et al., (2000) Nature 406:897-902) or the guanosine-binding protein Gαi2 (Rudolph et al., (1995) Nat. Genet. 10: 143-50) causes spontaneous colon cancer in rodents. Both of the aforementioned references are incorporated herein by reference. These studies indicate that potential causes other than alterations in the prototypical tumor suppressor genes and oncogenes could be involved in the etiology of human colon cancer.

A number of animal models for oral squamous cell carcinoma have been developed, including rat, mouse and hamster models. A hamster cheek pouch tumor model induced by the carcinogen 7,12-dimethylbenzanthracene remains one of the most common models (Baker (1986) Malignant neoplasms of the oral cavity. In: Otolaryngology—Head and Neck Surgery, Cummings et al. (eds.) pp. 1281-1343. St. Louis, Mo.: CV Mosby), but exhibits a number of differences from human oral cavity tumorigenesis. A recent mouse model using the carcinogen 4-nitroquinoline 1-oxide (4-NQO) has been developed which more closely simulates many aspects of human oral cavity and esophageal carcinogenesis (Tang et al. (2004) Clin. Cancer Res. 10: 301-313; incorporated herein by reference).

An animal model for multiple myeloma has been described (Garrett et al. (1997) Bone 20: 515-520; incorporated herein by reference), which uses a murine myeloma cell line 5TGM1 that causes lesions characteristic of human myeloma when injected into mice. Such lesions include severe osteolysis and the involvement of non-bone organs including liver and kidney. Mice inoculated with cultured 5TGM1 cells predictably and reproducibly develop disease, symptoms of which include the formation of a monoclonal gammopathy and radiologic bone lesions.

A number of animal models for the study of glioma exist, including an intracerebral rat glioma model (Sandström et al. (2004) Br. J. Cancer, 91: 1174-1180), and a murine model using injection of dog-derived J3T1 glioma cells (U.S. Pat. No. 6,677,155) (both incorporated herein by reference).

Animal models for the study of non-small cell lung cancer have been previously described, for example, by xenografting human tumors by subcutaneous transplantation of LC-6 human non-small cell lung cancer into BALB/C-nu/nu mice (Tashiro et al. (1989) Cancer Chemother Pharmacol 24, 187; herein incorporated by reference).

An animal model for the study of stomach cancer has been described which uses AZ-521 human stomach cancer xenografts in nude mice (Fukushima et. al. (2000) Biochem. Pharmacol. 59, 1227-1236; incorporated herein by reference).

Numerous animal models of AML have been previously described, including in rats (Blatt, J et al. (1991) Leuk Res 15:391-394), and SCID mice (Vey, N. et al. (2000) Clin. Cancer Res., 6:731-736) (both incorporated herein by reference).

A number of animal models used for the study of HCC have been described (Chisari et al., (1985) Science 230: 1157-1160; Babinet et al. (1985) Science 230: 1160-11; U.S. patent application Ser. No. 10/439,214) (all incorporated herein by reference). These references disclose the generation of transgenic mouse models of HCC by incorporating the HBV virus into the genome.

Animal models with experimental metastasis pattern resembling those frequently observed in human patients (Engebraaten & Fodstad, (1999) Int J Cancer. 82:219-25; incorporated herein by reference), which use MA-11 and MT-1, two estrogen and progesterone receptor-negative human breast cancer cell lines. Other models for breast cancer include U.S. patent application Ser. No. 10/410,207 (herein incorporated by reference). Alternatively, the ability of the compounds of the present invention to function as anti-breast cancer agents, either alone or in combination with other agents, can be demonstrated in vivo in carcinogen induced mammary tumors in wild type Sprague-Dawley Rats (Thompson H. J et al, Carcinogenesis, (1992) 13:1535- 1539; incorporated herein by reference).

A number of animal models for ovarian cancer are known in the art. For example, Connolly et al. ((2003) Cancer Research, 63, 1389-1397; incorporated herein by reference), discloses methods of developing epithelial ovarian cancer in mice by chimeric expression of the SV40 Tag under control of the MISIIR promoter. In another example, Liu et al. (Cancer Research 64, 1655-1663 (2004); incorporated herein by reference) have introduced human HRAS or KRAS oncogenes into immortalized human ovarian surface epithelial cells, which form subcutaneous tumors after injection into immunocompromised mice.

Numerous animal models for the study of prostate cancer are available. One murine model, using prostate cancer xenografts introduced into SCID mice, is disclosed in U.S. Pat. No. 6,756,036 (incorporated herein by reference). Alternatively, an orthotopic mouse model of metastatic prostate cancer can be used, as disclosed in U.S. patent application Ser. No. 10/417,727 (incorporated herein by reference).

### CHEMOTHERAPEUTIC AGENTS

The chemotherapeutic agents include alkylating agents, antimetabolites, natural products such as plant alkaloids and biologics. Alkylating agents bind covalently to DNA to inhibit DNA synthesis and stop cell growth. Suitable alkylating agents include, but are not limited to, nitrogen mustards such as chlorambucil, cyclphosphamide, estramustine, ifosfamide, mechlorethamine and melphalan, aziridine derivatives such as thiptepa, alkyl sulfonates such a busulfan and nitrosoureas, such as carmustine.

Antimetabolites are agents that block the biosynthesis or use of normal cellular metabolites. Similar to alkylating agents, antimetabolites inhibit DNA synthesis. However, antimetabolites are more effective against slower growing tumors than alkylating agents. Suitable antimetabolites include, but are not limited to, folate analogs such as methotrexate, purine analogs such as fludarabine, mercaptopurine and thioguanine, adenosine analogs such as cladribine and pentostatin and pyrimidine analogs such as capecitabine, cytarabine, depocyt, flosuridine and fluorouracil.

The third class of chemotherapeutic agents are natural products such as antitumor antibiotics. Suitable antitumor antibiotics include, but are not limited to, bleomycin, dactinomycin, daunorubicin, doxorubicin, doxil, epirubicin, idarubicin, mitomycin and mitoxantrone.

Other natural products include the vinca alkaloids which arrest cell division by preventing the formation of the mitotic spindle through disaggregation of microtubules. Suitable vinca alkaloids include, but are not limited to, vincristine, vinblastine, vinorelbine and vindesine. Taxanes are another type of natural product chemotherapeutic agent. Taxanes include, but are not limited to paclitaxel and docetaxel. The taxanes stabilize microtubules to inhibit mitotic spindle assembly to prevent cell division.

Biologics are yet another class of chemotherapeutic agents, and encompass monoclonal antibodies, soluble receptors, protein-chemotherapeutic conjugates, antisense oligonucleotides, and the like. Example of such agents include, Avastin® (bevacizumab), Campath® (alemtuzumab), Erbitux® (cetuximab), Herceptin® (trastuzumab), Rituxan™ (rituximab), Zevalin™ (ibritumomab tiuxetan), BEXXAR® (Tositumomab and I-131 tositumomab; monoclonal antibody targeting the CD20 antigen and radiolabeled version of the antibody), Mylotarg™ (gemtuzumab ozogamicin).

### RADIATION THERAPY

Radiation therapy can be used to treat almost every type of solid tumor, including brain, breast, cervix, larynx, lung, pancreas, prostate, skin, spine, stomach, uterus cancers, or soft tissue sarcomas. The appropriate dosage of radiation depends on a number of factors, including the type of cancer, type of radiation treatment, as well as proximity of radiation therapy to tissues and organs nearby that may be damaged by radiation, and tolerances of those tissues and organs to radiation. For example, radiation doses range from a low of 65 Gy to a high of 81 Gy for the treatment of prostate cancer, while for the treatment of solid epithelial tumors, the dosage can range between 50 Gy and 70 Gy. In contrast, lymphomas typically receive lower doses, ranging between 20 to 40 Gy in daily doses.

Radiation therapies which are suitable for use in the combination treatments described herein, include the use of a) external beam radiation; and b) a radiopharmaceutical agent which comprises a radiation-emitting radioisotope.

### EXTERNAL BEAM RADIATION

External beam radiation therapy for the treatment of cancer uses a radiation source that is external to the patient, typically either a radioisotope, such as ⁶⁰Co, ¹³⁷Cs, or a high energy x-ray source, such as a linear accelerator. The external source produces a collimated beam directed into the patient to the tumor site. External-source radiation therapy avoids some of the problems of internal-source radiation therapy, but it undesirably and necessarily irradiates a significant volume of non-tumorous or healthy tissue in the path of the radiation beam along with the tumorous tissue.

The adverse effect of irradiating of healthy tissue can be reduced, while maintaining a given dose of radiation in the tumorous tissue, by projecting the external radiation beam into the patient at a variety of “gantry” angles with the beams converging on the tumor site. The particular volume elements of healthy tissue, along the path of the radiation beam, change, reducing the total dose to each such element of healthy tissue during the entire treatment.

The irradiation of healthy tissue also can be reduced by tightly collimating the radiation beam to the general cross section of the tumor taken perpendicular to the axis of the radiation beam. Numerous systems exist for producing such a circumferential collimation, some of which use multiple sliding shutters which, piecewise, can generate a radio-opaque mask of arbitrary outline.

A new method of external radiotherapy, called conformal radiotherapy or three-dimensional conformal radiotherapy, can also be used to treat tumors that, in the past, were considered too close to a vital organ or tissue to permit effective radiotherapy. The complex process of conformal radiotherapy begins with the creation of a three-dimensional reconstruction of a patient's tumors and normal adjacent anatomy. The 3-D computer images thus developed are used to deliver highly focused, or “conformed” radiotherapy to the tumor while sparing normal adjacent tissue, resulting in overall higher dosage of radiation than previously permitted, while causing less harm to proximal tissues and organs.

### RADIOPHARMACEUTICAL AGENTS

A “radiopharmaceutical agent”, as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

As used herein, a “metallic radioisotope” is any suitable metallic radioisotope useful in a therapeutic or diagnostic procedure in vivo or in vitro. Suitable metallic radioisotopes include, but are not limited to: Actinium-225, Antimony-124, Antimony-125, Arsenic-74, Barium-103, Barium-140, Beryllium-7, Bismuth-206, Bismuth-207, Bismuth212, Bismuth213, Cadmium-109, Cadmium-15m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium153, Gadolinium-157 Gold-195, Gold-199, Hafnium-175, Hafiium-175-181, Holmium-166, Indium-110, Indium-111, Iridium-192, Iron 55, Iron-59, Krypton85, Lead-203, Lead-210, Lutetium-177, Manganese-54, Mercury-197, Mercury203, Molybdenum-99, Neodymium-147, Neptunium-237, Nickel-63, Niobium95, Osmium-185+191, Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-103, Ruthenium-105, Ruthenium-106, Samarium-153, Scandium-44, Scandium-46, Scandium-47, Selenium-75, Silver-110m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, Zirconium-89, and Zirconium-95.

As used herein, a “non-metallic radioisotope” is any suitable nonmetallic radioisotope (non-metallic radioisotope) useful in a therapeutic or diagnostic procedure in vivo or in vitro. Suitable non-metallic radioisotopes include, but are not limited to: Iodine-131, Iodine-125, Iodine-123, Phosphorus-32, Astatine-211, Fluorine-18, Carbon-11, Oxygen-15, Bromine-76, and Nitrogen-13.

Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects.

It is preferred that the physical half-life of the therapeutic radioisotope be similar to the biological half-life of the radiopharmaceutical at the tumor site. For example, if the half-life of the radioisotope is too short, much of the decay will have occurred before the radiopharmaceutical has reached maximum target/background ratio. On the other hand, too long a half-life would cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope has to be long enough to allow adequate time for manufacturing, release, and transportation.

Other practical considerations in selecting a radioisotope for a given application in tumor therapy are availability and quality. The purity has to be sufficient and reproducible, as trace amounts of impurities can affect the radiolabeling and radiochemical purity of the radiopharmaceutical.

The target receptor sites in tumors are typically limited in number. As such it is preferred that the radioisotope have high specific activity. The specific activity depends primarily on the production method. Trace metal contaminants must be minimized as they often compete with the radioisotope for the chelator and their metal complexes compete for receptor binding with the radiolabeled chelated agent.

The type of radiation that is suitable for use in the methods of the present invention can vary. For example, radiation can be electromagnetic or particulate in nature. Electromagnetic radiation useful in the practice of this invention includes, but is not limited to, x-rays and gamma rays. Particulate radiation useful in the practice of this invention includes, but is not limited to, electron beams (beta particles), protons beams, neutron beams, alpha particles, and negative pi mesons. The radiation can be delivered using conventional radiological treatment apparatus and methods, and by intraoperative and stereotactic methods. Additional discussion regarding radiation treatments suitable for use in the practice of this invention can be found throughout Steven A. Leibel et al., Textbook of Radiation Oncology (1998) (publ. W. B. Saunders Company), and particularly in Chapters 13 and 14. Radiation can also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods can be used in the practice of this invention.

For tumor therapy, both α and β-particle emitters have been investigated. Alpha particles are particularly good cytotoxic agents because they dissipate a large amount of energy within one or two cell diameters. The β-particle emitters have relatively long penetration range (2-12 mm in the tissue) depending on the energy level. The long-range penetration is particularly important for solid tumors that have heterogeneous blood flow and/or receptor expression. The β-particle emitters yield a more homogeneous dose distribution even when they are heterogeneously distributed within the target tissue.

### HORMONAL ABLATION THERAPY

Hormonal ablation can be used to treat certain cancers, such as breast cancer. Specifically, ovarian ablation/suppression and tamoxifen are currently accepted adjuvant endocrine therapies, for premenopausal breast cancer. Methods of permanently ablating ovarian function include surgical oophorectomy and radiation-induced ovarian failure; medical castration with luteinizing hormone-releasing hormone analogues is a reversible approach.

### 24-HYDROXYLASE INHIBITORS

The activation and inactivation pathways for vitamin D are complex. Vitamin D₃ is synthesized in the skin and then becomes hydroxylated at the C-25 position by 25-hydroxylase (also known as CYP27) in the liver. The key enzyme in synthesizing the most active metabolite, 1,25-(OH)₂ vitamin D₃, is 25-hydroxyvitamin D 1-alpha-hydroxylase (CYP27B1), which is primarily expressed in the kidney. Both 1,25-(OH)₂ vitamin D₃ and the less active metabolite 25(OH)D₃ are converted to the inactive forms 1,24,25(OH)₃ vitamin D₃, and 24,25(OH)₂ vitamin D₃, respectively, by 24-hydroxylase (also known as CYP24) in kidney and in skin. According to the invention, the administration of a 24-hydroxylase inhibitor reduces the breakdown of 1,25-(OH)₂ vitamin D₃. In some embodiments the administration of a 24-hydroxylase inhibitor does not result in hypercalcemia.

An “inhibitor of 24-hydroxylase” is any chemical compound that has the property of reducing the enzyme activity of CYP24, also known as “vitamin D 24-hydroxylase” or “24-hydroxylase.” Important physiological substrates for this enzyme, which is normally found in the inner mitochondrial membrane of proximal renal tubule cells, epidermal keratinocytes, and other cells, are 1,25-(OH)₂ vitamin D₃ and 25-OH vitamin D₃, which it converts to the less active metabolites 1,24,25-(OH)₃ vitamin D₃ and 24,25-(OH)₂ vitamin D₃, respectively. An “inhibitor of 24-hydroxylase” can reduce the rate of the enzyme reaction catalyzed by 24-hydroxylase by any amount, for example, by a statistically significant amount, by at least 1%, at least 2%, at least 3%, at least 5%, at least 10%, at least 15%, at least 20%, at least 30%, at least 50%, at least 100%, at least 2-fold, at least 3-fold, at least 5-fold, at least 10-fold, at least 100-fold, or at least 1000-fold or more. Inhibition can be by any mechanism, for example, by competitive, uncompetitive, or noncompetitive inhibition.

Azoles are potent inhibitors of cytochrome P450 enzymes which directly bind to heme iron via a single electron pair from the azole nitrogen. Further, azoles interact with the substrate binding pocket. See Poulos, Pharm. Res. 5:67-75 (1988). Thus, azole inhibitors of CYP enzymes can block both oxygen and substrate binding and provide high-affinity binding. Examples of azole drugs are the antifungals ketoconazole, clotrimazole, itraconazole, and fluconazole. While these are potent CYP inhibitors, they may not possess adequate selectivity if they are capable of binding to heme iron in different CYP enzymes.

As used herein, an “azole” is a compound comprising a five-membered heterocyclic ring with two double bonds, which ring also contains an atom of nitrogen and at least one other noncarbon atom, such as oxygen, sulphur, or another nitrogen atom. Preferred azoles for use in the invention are those which are capable of inhibiting CYP24, or 24-hydroxylase. More preferred are azole compounds which selectively inhibit CYP24, i.e., compounds which have a lower IC₅₀ value for CYP24 than for other enzymes, including CYP27B, which is responsible for the final step in the synthesis of 1,25-(OH)₂ vitamin D₃. Preferred azoles of the invention are those which have a bulky group attached to the C atom which is alpha to the azole group. A “bulky group” in this context is a cyclic or branched alkyl substituent. For example, the bulky group can be a phenyl, naphthyl, thienyl or pyridyl substituent; or a phenyl, naphthyl, thienyl or pyridyl substituent monosubstituted by halogen, (C₁₋₄)alkoxy, (C₁₋₄)alkyl, di-(C₁₋₄) alkylamino or cyano.

Schuster et al., J. Cell. Biochem. 88:372-380 (2003) (hereby incorporated by reference in its entirety) have determined structure-activity relationships for selective and potent 24-hydroxylase inhibitors. Pharmacophore models were built by superimposing a large group of inhibitors of CYP24 and CYP27B1. A program called DISCO (DIStance COmparison, Tripos), a module of the computational SYBYL software, was used to obtain information on the shape, size, and electrostatic properties of the active site of CYP24. Schuster et al. determined that selectivity for CYP24 was achieved by positioning bulky substituents in the α-position relative to the azole. On the other hand, bulky substituents in the β-position to the azole favored selectivity for CYP27B1. The active sites of both CYP24 and CYP27B1 shared several common features, including a similar large size and the presence of at least two hydrophobic regions. The location of the hydrophobic regions was different, which led to the principle that substitution with large bulky groups in the α-position to the azole favors CYP24 binding whereas large bulky groups in β-position to the azole favors binding to CYP27B1.

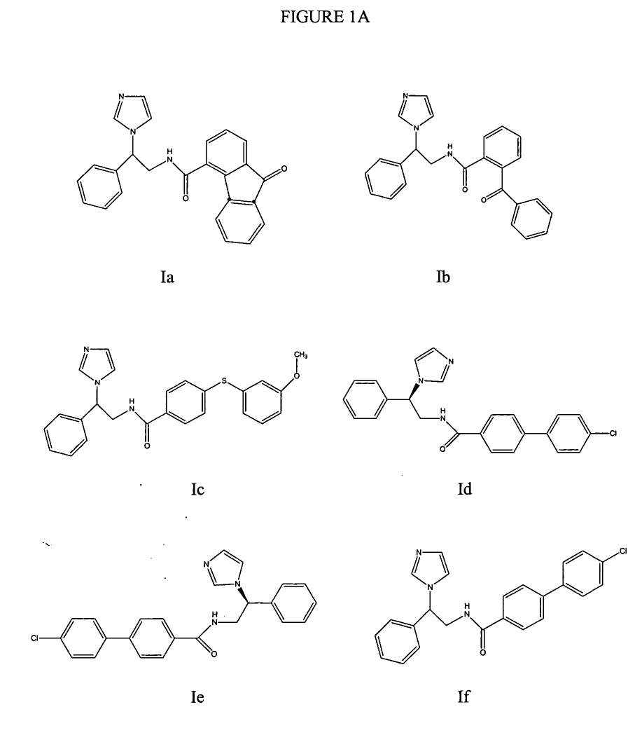

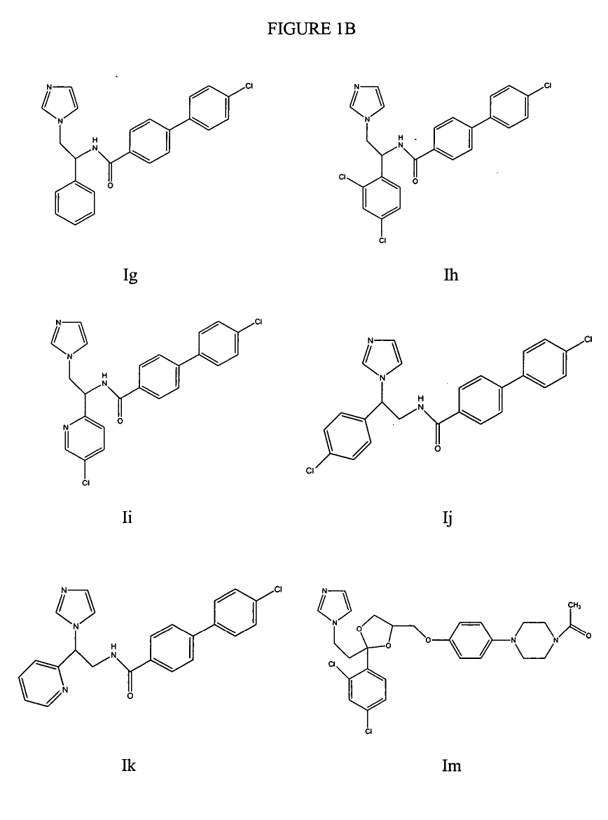

Several specific compounds identified by Schuster et al., J. Cell. Biochem. 88:372-380 (2003) are potent inhibitors of 24-hydroxylase. These include (R)-SDZ-286907, (R)-SDZ-287871, (R)-VAB636, (S)-SDZ-285428, and (R)-VID400 (2-(R)-4′-Chlorobiphenyl-4-carboxylic acid (2-imidazol-1-yl-2-phenyl-ethyl)-amide) (see FIG. 1).

In a specific embodiment, the 24-hydroxylase inhibitor compounds are represented by the structural Formula I or a pharmaceutically acceptable salt, solvate, hydrate, ester, or isomer thereof, 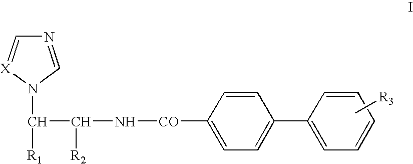
wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄)alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

The acylated aminoalkanimidazoles and aminoalkantriazoles of Formula I are fully described in U.S. Pat. No. 5,622,982 to Schuster et al., the entire content of which is hereby incorporated by reference.

In another embodiment, the 24-hydroxylase inhibitor is represented by the structural Formula II or a pharmaceutically acceptable salt, solvate, hydrate, ester, or isomer thereof, 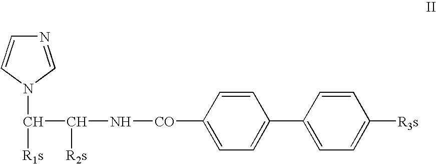
wherein:
R_{1S} is phenyl, phenyl monosubstituted by halogen, or 1-naphtyl, and R_{2S} is hydrogen; or
R_{1S} is hydrogen and R_{2S} is pyridyl or 2-(5-chloro)pyridyl; and
R_{3S} is halogen, (C₁₋₄) alkoxy.

The compounds of Formula II are fully described in U.S. Pat. No. 5,622,982.

Vitamin D analogs also can be effective inhibitors of 24-hydroxylase. The structure of 1,25-(OH)₂ vitamin D₃, also known as calcitriol, is depicted below.

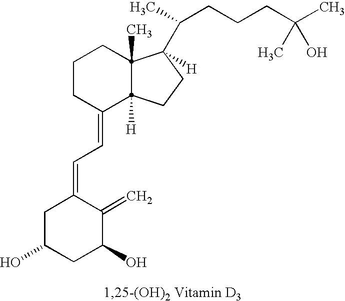

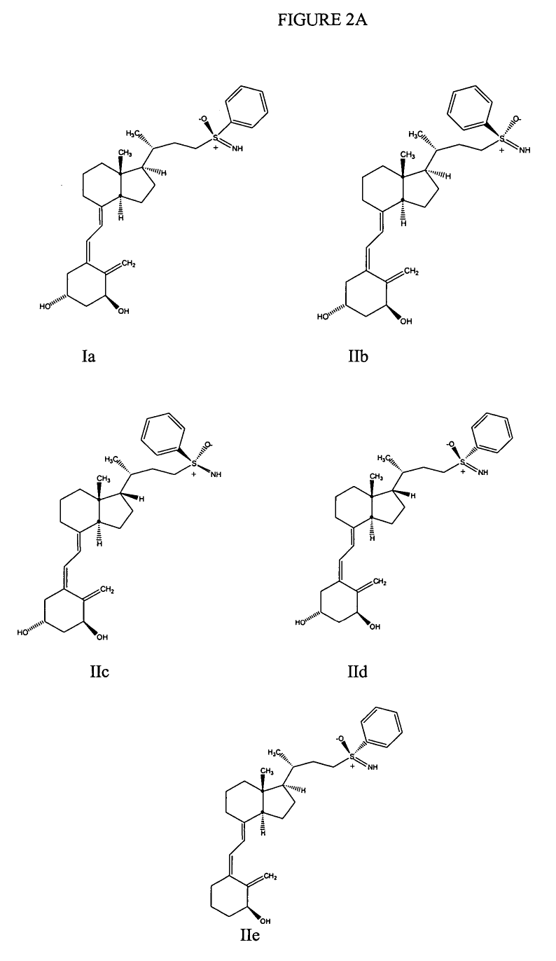

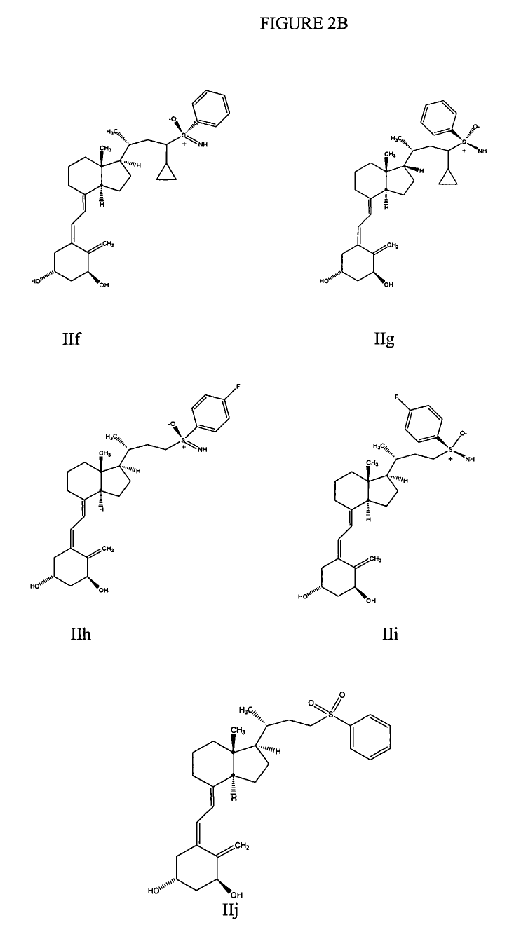

As used herein, a “structural analog of 1,25-(OH)₂ vitamin D₃” is a compound that retains the ring structures and backbone of 1,25-(OH)₂ vitamin D₃, but wherein one or more of the substituents attached thereto (e.g., H, OH, CH₂, or CH₃) have been removed or replaced with other substituents, or wherein the terminal C atom of the backbone (C₂₅) has been replaced with one or more substituents. Preferably, structural analogs of 1,25-(OH)₂ vitamin D₃ of the instant invention have the property of inhibiting 24-hydroxylase or are resistant to degradation by 24-hydroxylase. For example, Kahraman et al. (J. Med.Chem. 47:6854-6863 (2004); incorporated herein by reference) have described a set of 24-sulfoximine derivatives of 1,25-(OH)₂ vitamin D₃. Representative examples with good inhibitory potency for 24-hydroxylase are depicted in FIG. 2.

Thus, in certain embodiments the inhibitor is a structural analog of 1,25-(OH)₂ vitamin D₃. For example, the inhibitor may be represented by Formula IV:

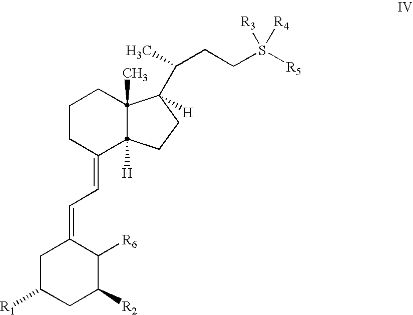

wherein R₁ and R₂ are each independently selected from the group consisting of hydrogen, OR′, —C(O)H, and —C(O)R′;

wherein R′ is selected from the group consisting of a C1 to C₆ alkyl, a cycloalkyl, phenyl, an alkylaryl, an arylalkyl, and a heteroaryl; each of which can be optionally substituted with at least one halogen, thiol, mercapto, hydroxyl, or amino group;

wherein R₃, R₄ and R₅ are each independently selected from the group consisting of hydrogen, hydroxyl, oxy, imine, phenyl, a C1 to C₆ alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and

wherein R₆ is hydrogen, ═CH₂ or a C₁ to C₆ alkyl, alkenyl, cycloalkyl, or cycloalkenyl, each of which can be optionally substituted with at least one halogen, thiol, mercapto, hydroxyl, or amino group;

or a pharmaceutically acceptable salt, hydrate, solvate, ester, or isomer thereof.

Structure activity relationships for 24-sulfoximine analogs of 1,25-(OH)2 vitamin D3 have been described by Kahraman et al. The most potent compound appears to be the phenyl sulfoximine shown in Formula V:

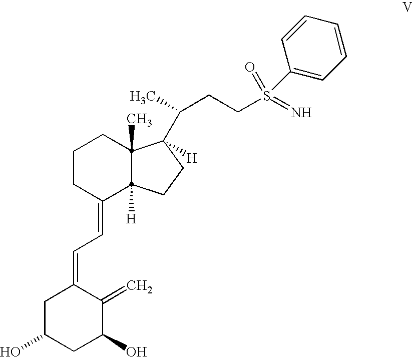

Only slightly less potent is the 4-fluorophenyl sulfoximine shown in Formula VI: 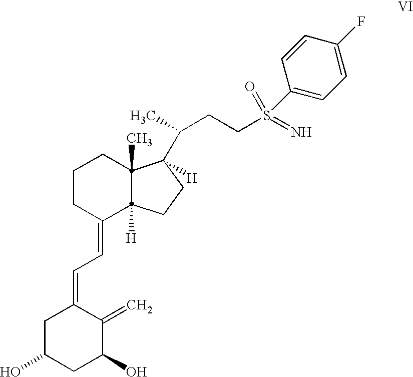
In general, the stereochemical configuration at the 24-sulfur atom is significant, with the 24-(S) configuration being more potent than the 24-(R) configuration. 24-sulfone analogs are less potent that 24-sulfoximines, and 22-ene analogs are much less potent.

The 24-hydroxylase inhibitors described above intended to be merely illustrative. It is understood that further such compounds can be readily obtained using the principles outlined herein and in the cited references.

24-Hydroxylase Inhibition Assay

Inhibition of 24-hydroxylase activity from human keratinocytes can be performed according to Schuster et al., J. Cell. Biochem. 88:372-380 (2003). Briefly, confluent cultures of human keratinocytes are isolated from adult skin in serum-free keratinocyte growth medium (KGM, Clonetics) with physiological levels of [26,27-³H]-25(OH)vitamin D₃ (10-20 nM; 15-17 Ci/mmol, Amersham) in the absence or presence of varying concentrations (0 to 10 μM) of a 24-hydroxylase inhibitor. 24-hydroxylase activity was determined after 1 h incubation as the rate of substrate conversion to 1-hydroxylated metabolites (1,25(OH)₂D₃ and 1,25(OH)₂-3-epi-D₃). Formation of individual metabolites can be determined by high performance liquid chromatography of organic incubation extracts on Zorbax-Sil using a nonlinear gradient of 97:3-85:15% n-hexane: 2-propanol), using a radioactivity detector (Radiomatic 500TR, Canberra) to detect and quantify radioactive peaks. IC₅₀ values can be obtained from plots of enzyme activity versus inhibitor concentration.

### DOSING AND ADMINISTRATION

Subject, as used herein, refers to animals such as mammals, including, but not limited to, primates (e.g., humans), cows, sheep, goats, horses, pigs, dogs, cats, rabbits, guinea pigs, rats, mice or other bovine, ovine, equine, canine, feline, rodent or murine species. In a preferred embodiment, the mammal is a human.

As used herein, treating and treatment refer to partially or totally inhibiting formation of, or otherwise treating (e.g., reversing or inhibiting the further development of) cancer such as tumors, neoplasms, carcinomas, sarcomas, leukemias, lymphomas and the like.

As used herein, therapeutically effective amount refers to an amount sufficient to elicit the desired biological response. In the present invention, the desired biological response is partially or totally inhibiting formation of, or otherwise treating (e.g., reversing or inhibiting the further development of) cancer such as tumors, neoplasms, carcinomas, sarcomas, leukemias, lymphomas and the like.

A therapeutically effective amount can be achieved in the method or pharmaceutical composition of the invention employing a first amount of a 24-hydroxylase inhibitor and a second amount of a suitable cancer therapeutic. In one embodiment, the 24-hydroxylase inhibitor and the suitable cancer therapeutic are each administered in a therapeutically effective amount (i.e., each in an amount which would be therapeutically effective if administered alone). In another embodiment, the 24-hydroxylase inhibitor and the suitable cancer therapeutic are each administered in an amount which alone does not provide a therapeutic effect (a sub-therapeutic dose). In yet another embodiment, the 24-hydroxylase inhibitor can be administered in a therapeutically effective amount, while the suitable cancer therapeutic is administered in a sub-therapeutic dose. In still another embodiment, the 24-hydroxylase inhibitor can be administered in a sub-therapeutic dose, while the suitable cancer therapeutic is administered in a therapeutically effective amount. It is understood that the method of coadministration of a first amount of a 24-hydroxylase inhibitor and a second amount of a suitable cancer therapeutic can result in an enhanced or synergistic therapeutic effect, wherein the combined effect is greater than the additive effect that would result from separate administration of the first amount of the 24-hydroxylase inhibitor and the second amount of the suitable cancer therapeutic. A synergistic effect can be, for example, an increase of 3-fold, 10-fold, 100-fold or greater therapeutic effect than the sum of the therapeutic effects expected from administering each agent separately. The greater therapeutic effect can be manifested in a variety of ways, for example, greater reduction in tumor size, more rapid reduction in tumor size, reduced morbidity or mortality, or longer time until recurrance of the tumor.

The presence of a synergistic effect can be determined using suitable methods for assessing drug interaction. Suitable methods include, for example, the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Pharmaceutically acceptable carrier, includes pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

Pharmaceutically acceptable carriers can be aqueous or non-aqueous solvents. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media.

The compounds for use in the method of the invention can be formulated for administration by any suitable route, such as for oral or parenteral, for example, transdermal, transmucosal (e.g., sublingual, lingual, (trans)buccal), vaginal (e.g., trans- and perivaginally), (intra)nasal and (trans)rectal), subcutaneous, intramuscular, intradermal, intra-arterial, intravenous, inhalation, and topical administration.

Suitable compositions and dosage forms include tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays, dry powders or aerosolized formulations.

It is preferred that the compounds are orally administered. Suitable oral dosage forms include, for example, tablets, capsules or caplets prepared by conventional means with pharmaceutically acceptable excipients such as binding agents (e.g., polyvinylpyrrolidone or hydroxypropylmethylcellulose); fillers (e.g., lactose, microcrystalline cellulose or calcium phosphate); lubricants (e.g., magnesium stearate, talc or silica); disintegrates (e.g., sodium starch glycollate); or wetting agents (e.g., sodium lauryl sulphate). If desired, the tablets can be coated, e.g., to provide for ease of swallowing or to provide a delayed release of active, using suitable methods. Liquid preparation for oral administration can be in the form of solutions, syrups or suspensions. Liquid preparations (e.g., solutions, suspensions and syrups) are also suitable for oral administration and can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

As used herein, the term “pharmaceutically acceptable salt” refers to a salt of a compound to be administered prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

The 24-hydroxylase inhibitor compounds disclosed can be prepared in the form of their hydrates, such as hemihydrate, monohydrate, dihydrate, trihydrate, tetrahydrate and the like and as solvates.

It is understood that 24-hydroxylase inhibitor compounds can be identified, for example, by screening libraries or collections of molecules using suitable methods. Another source for the compounds of interest are combinatorial libraries which can comprise many structurally distinct molecular species. Combinatorial libraries can be used to identify lead compounds or to optimize a previously identified lead. Such libraries can be manufactured by well-known methods of combinatorial chemistry and screened by suitable methods.

As used herein, continuous dosing refers to the chronic administration of a selected active agent.

As used herein, as-needed dosing, also known as “pro re nata” “pm” dosing, and “on demand” dosing or administration is meant the administration of a therapeutically effective dose of the compound(s) at some time prior to commencement of an activity wherein suppression of a lower urinary tract disorder would be desirable.

Administration can be immediately prior to such an activity, including about 0 minutes, about 10 minutes, about 20 minutes, about 30 minutes, about 1 hour, about 2 hours, about 3 hours, about 4 hours, about 5 hours, about 6 hours, about 7 hours, about 8 hours, about 9 hours, or about 10 hours prior to such an activity, depending on the formulation.

In a particular embodiment, drug administration or dosing is on an as-needed basis, and does not involve chronic drug administration. With an immediate release dosage form, as-needed administration can involve drug administration immediately prior to commencement of an activity wherein suppression of the symptoms of overactive bladder would be desirable, but will generally be in the range of from about 0 minutes to about 10 hours prior to such an activity, preferably in the range of from about 0 minutes to about 5 hours prior to such an activity, most preferably in the range of from about 0 minutes to about 3 hours prior to such an activity.

Suitable dosing ranges for 24-hydroxylase inhibitors can be, for example, from about 100 micrograms to about 2 g per day, for example, from about 200 micrograms to about 1 g per day, such as from about 300 micrograms to about 750 mg per day, or for example, from about 400 micrograms to about 600 mg per day.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### COADMINISTRATION

In practicing the methods of the invention, coadministration refers to administration of a first amount of the 24-hydroxylase inhibitor and a second amount of a suitable cancer therapeutic, wherein the first and second amounts together comprise a therapeutically effective amount to treat cancer in a subject in need of treatment. Coadministration encompasses administration of the first and second amounts of the compounds of the coadministration in an essentially simultaneous manner, such as in a single pharmaceutical composition, for example, capsule or tablet having a fixed ratio of first and second amounts, or in multiple, separate capsules or tablets for each. In addition, such coadministration also encompasses use of each compound in a sequential manner in either order. When coadministration involves the separate administration of the first amount of the 24-hydroxylase inhibitor and the second amount of the suitable cancer therapeutic the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, the 24-hydroxylase inhibitor and the suitable cancer therapeutic can be administered in any order within about 24 hours of each other, within about 16 hours of each other, within about 8 hours of each other, within about 4 hours of each other, within about 1 hour of each other or within about 30 minutes of each other.

The therapeutically effective amount of a first amount of the 24-hydroxylase inhibitor and a second amount of a suitable cancer therapeutic in combination will depend on the age, sex and weight of the patient, the current medical condition of the patient and the nature of the cancer being treated. The skilled artisan will be able to determine appropriate dosages depending on these and other factors. The ratio of 24-hydroxylase inhibitor to any single cancer therapeutic can be, for example, in the range of about 1:1000, 1:100, 1:50, 1:10, 1:1, 10:1, 50:1, 100:1, or 1000:1 on a weight basis. A 24-hydroxylase inhibitor may be co-administered with one or more cancer therapeutics, including one or more chemotherapeutic agents and radiation. Calcitriol, or another vitamin D metabolite or analog, is considered a possible chemotherapeutic agent which can be co-administered with a 24-hydroxylase inhibitor and, optionally, one or more further cancer therapeutics.

### STEREOCHEMISTRY

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture.

Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When compounds of the present invention contain one chiral center, the compounds exist in two enantiomeric forms and the present invention includes either or both enantiomers and mixtures of enantiomers, such as the specific 50:50 mixture referred to as a racemic mixture. The enantiomers can be resolved by methods known to those skilled in the art, for example by formation of diastereoisomeric salts which may be separated, for example, by crystallization (See, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least about 99% enantiomeric excess.

When a compound of the present invention has two or more chiral carbons, it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Anti-Proliferative Activity of 24-Hydroxylase Inhibitors in Combination with Docetaxel in a Prostate Cancer Cell Line

The potential for (S)-SDZ-285428 to augrnent the activity of a commonly used chemotherapeutic agent, docetaxel, against a prostate cancer cell line, PC-3 was evaluated. A fixed concentration of (S)-SDZ-285428 was added to serial dilutions of a constant 1:42 molar ratio of calcitriol:docetaxel and the effects of the combinations on PC-3 cell growth were compared with the same serial dilutions of calcitriol:docetaxel in the absence of (S)-SDZ-285428. A molar ratio of calcitriol-to-docetaxel of 1:42 was used to ensure that some basal amount of calcitriol was present under the experimental conditions, and for detecting synergistic effects of additional reagents should such effects be present.

The experiment was described essentially as described in detail (Mol. Can. Therapeutics, Vol 1; 821 (2002)). Cells were incubated at 37° C., 5% CO₂ and allowed to attach for 24 hours. Following attachment, the culture medium was removed and replaced with experimental medium containing calcitriol:docetaxel either with or without added (S)-SDZ-285428 (at a final concentration of 200 nM). Following 48 to 72 hours of growth, the cell density was determined. Growth rates were compared against a control without docetaxel, calcitriol, and (S)-SDZ-285428. Data were expressed as a percentage relative to the control. A Combination Index (CI) was calculated by isobologram analysis according to previously published methods (Mol. Can. Therapeutics, Vol 1; 821 (2002). A Combination Index of less than 1.0 indicates synergy.

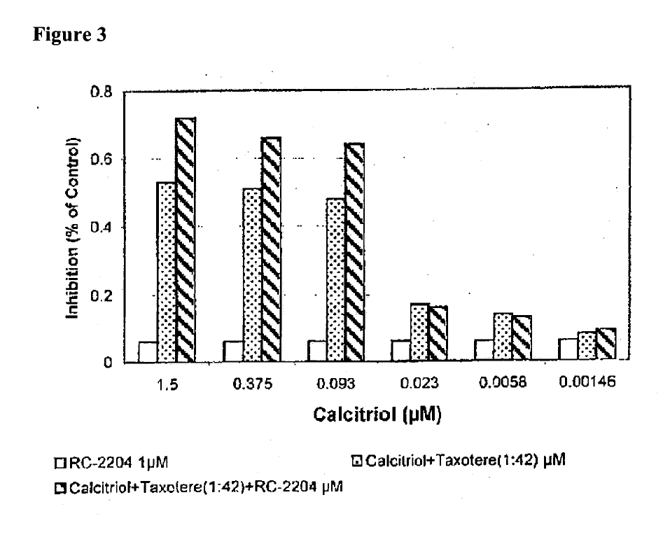

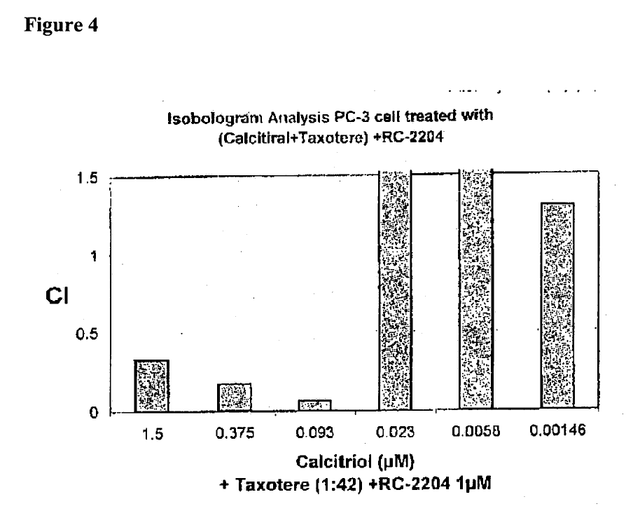

As shown in FIG. 3, the growth inhibitory effects of calcitriol:docetaxel were heightened in the presence of (S)-SDZ-285428when compared with no addition of (S)-SDZ-285428. In addition, the added inhibitory effect was synergistic. The synergistic effect is better illustrated in the isobologram of FIG. 4, where CI values of less than 1.0 were observed in treatments in which the concentration of calcitriol:docetaxel was greater than 0.093:3.906 μM. In conclusion, (S)-SDZ-285428 demonstrated synergistic effects in combination with calcitriol:docetaxel.

### Example 2

### Effects of 24-Hydroxylase Inhibitor on B-Cell Lymphomas

Human RL B-lymphoma cells (Beckwith et al. (1990) J. Nat. Cancer Inst. 82, 501) are maintained in vitro by passage in growth medium. The cells are washed thoroughly in PBS to remove culture components. SCID mice are injected with one million human lymphoma cells via the tail vein in 100 microliters (μl). 24-hydroxylase inhibitor treatment is initiated the following day by daily intraperitoneal (i.p.) injection of 3 mg/kg 24-hydroxylase inhibitor (VID400), alone or in combination with doxorubicin at 5 mg/kg, or vehicle. Mice are monitored for survival and significant morbidity. Mice that lose greater than 20% of their initial body weight, as well as mice that exhibit other symptoms such as hind limb paralysis, are sacrificed. Depending on the lymphoma cell line employed, the untreated mice typically die within 3 to 6 weeks. Both doxorubicin and doxorubicin+24-hydroxylase inhibitor treatments are expected to show significant inhibition in tumor growth, with doxorubicin+24-hydroxylase inhibitor treatment expected to show the highest growth inhibition.

### Example 3

### Effects of a 24-Hydroxylase Inhibitor in a Mouse Ovarian Carcinoma Model

The efficacy of a 24-hydroxylase inhibitor, alone or in combination with a chemotherapeutic agent is tested in a mouse syngeneic model of ovarian carcinoma using methods as described in Zhang et al., (Am. J. of Pathol. (2002) 161:2295-2309). Briefly, using retroviral transfection and fluorescence-activated cell sorting, a C57BL6 murine ID8 ovarian carcinoma cell line is generated that stably overexpresses the murine VEGF164 isoform and the enhanced green fluorescence protein (GFP). The retroviral construct containing VEGF164 and GFP cDNAs is then transfected into BOSC23 cells. The cells are analyzed by FACS cell sorting, and cells showing high GFP fluorescence are identified and isolated. The ID8 VEGF164/GFP transfected cells are cultured to subconfluence and prepared in a single-cell suspension in phosphate buffer saline (PBS) and cold MATRIGEL (BD Biosciences, Bedford, Mass.). Six to eight week old female C57BL6 mice are injected subcutaneously in the flank with 5×10⁶ transfected or untransfected (control) cells. Animals are sacrificed eight weeks after inoculation and evaluated for tumor growth. Mice are treated with a 24-hydroxylase inhibitor (VID 400/RC-8800) beginning 3-14 days following tumor implantation. Mice are treated in the following groups, with 5 mice for each group:

| | |
|---|---|
| Group 1: | Vehicle |
| Group 2: | 24-hydroxylase inhibitor only (2 mg/kg) |
| Group 3: | 24-hydroxylase inhibitor (2 mg/kg) + Cisplatin (2 mg/kg) |
| Group 4: | Cisplatin only (2 mg/kg) |
| Group 5: | 24-hydroxylase inhibitor (2 mg/kg) + Cisplatin (6 mg/kg) |
| Group 6: | Cisplatin only (6 mg/kg) |

The agents are administered on a daily basis for 14 days. Animals are monitored daily for body weight, and significant morbidity. Tumor growth plots are expected to show significant delay in Groups 2-6 when compared with vehicle control, with combinations of 24-hydroxylase inhibitor and cisplatin expected to delay longer than cisplatin alone (at the same dosage), or 24-hydroxylase alone. Likewise mice treated with combinations of 24-hydroxylase inhibitor and cisplatin are expected to maintain body weight significantly more than vehicle or single therapy controls of the same dosage.

### Example 4

### Effects of 24-Hydroxylase Inhibitors in a Mouse Colorectal Tumor Model

The effects of 24-hydroxylase inhibitors in a colorectal mouse model are tested as described in Yao et al., (Cancer Res. (2003) 63:586-592; incorporated herein by reference). In this model, MC-26 mouse colon tumor cells are implanted into the splenic subcapsule of BALB/c mice. Mice are administered 24-hydroxylase inhibitor daily at different dosages, in groups of five animals for 3-14 days following when tumor engraftment and growth rate is established. Groups are as follows:

| | |
|---|---|
| Group 1: | Vehicle |
| Group 2: | 24-hydroxylase inhibitor at 3 mg/kg |
| Group 3: | 24-hydroxylase inhibitor at 10 mg/kg |
| Group 4: | 24-hydroxylase inhibitor at 30 mg/kg |

The efficacy of the 24-hydroxylase inhibitor treatments in prolonging survival or promoting a tumor response is evaluated using body weight measurement, tumor growth delay plots, and morbidity.

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred and non-limiting embodiments only, and that in general numerous equivalent methods and techniques may be employed to achieve the same result. While this invention has been particularly shown and described with references to preferred embodiments thereof, it will be understood by those skilled in the art that various changes in form and details may be made therein without departing from the scope of the invention encompassed by the appended claims.

What is claimed is:

1. A method for treating cancer in a subject in need thereof comprising administering to said subject:
i) a first amount of a 24-hydroxylase inhibitor; and
ii) a second amount of a suitable cancer therapeutic
wherein the first and second amounts together comprise a therapeutically effective amount.

2. The method of claim 1, wherein the suitable cancer therapeutic is a chemotherapeutic.

3. The method of claim 2, wherein the chemotherapeutic is selected from the group consisting of: paclitaxel, docetaxel, an antitumor antibiotic, an alkylating agent, a plant alkaloid, a biologic or a combination thereof.

4. The method of claim 1, wherein the suitable cancer therapeutic is radiation therapy.

5. The method of claim 4, wherein the radiation therapy is selected from the group consisting of: external beam radiation, radiopharmaceutical agent or a combination thereof.

6. The method of claim 1, wherein the suitable cancer therapeutic is hormonal ablation.

7. The method of claim 6, wherein the hormonal ablation therapy is selected from the group consisting of ovarian ablation, ovarian suppression, tamoxifen, surgical oophorectomy, radiation-induced ovarian failure, medical castration with luteinizing hormone-releasing hormone analogues or a combination thereof.

8. The method of claim 1, wherein the 24-hydroxylase inhibitor is administered orally.

9. The method of claim 1, wherein the 24-hydroxylase inhibitor is represented by the structural Formula I: 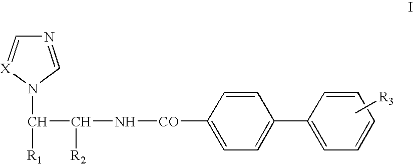
or a pharmaceutically acceptable salt, solvate or hydrate thereof, wherein:
R₁ is phenyl, naphthyl, thienyl or pyridyl, or phenyl, naphthyl, thienyl or pyridyl monosubstituted by halogen, (C₁₋₄)alkoxy, (C₁₋₄)alkyl, di-(C₁₋₄)alkylamino or cyano and R₂ is hydrogen; or
R₁ is hydrogen and R₂ is pyridyl or 2-(5-chloro)pyridyl;
R₃ is hydrogen, halogen, (C₁₋₄) alkyl, (C₁₋₄) alkoxy, cyano, (C₁₋₄) alkoxycarbonyl, (C₁₋₄) alkylcarbonyl, amino or di-(C₁₋₄) alkylamino; and
X is CH or N.

10. The method of claim 1, wherein the 24-hydroxylase inhibitor is represented by the structural Formula II: 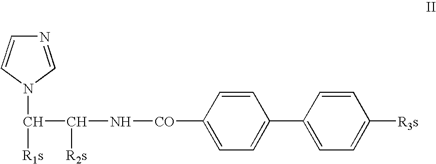
or a pharmaceutically acceptable salt, solvate or hydrate thereof, wherein:
R_{1S} is phenyl, phenyl monosubstituted by halogen, or 1-naphtyl, and R_{2S} is hydrogen; or
R_{1S} is hydrogen and R_{2S} is pyridyl or 2-(5-chloro)pyridyl; and
R_{3S} is halogen, (C₁₋₄) alkoxy.

11. The method of claim 1, wherein the 24-hydroxylase inhibitor is selected from the group consisting of structures 1d and 1e of FIG. 1, or a pharmaceutically acceptable salt, solvate or hydrate thereof.

12. The method of claim 1, wherein the cancer is selected from the group consisting of colorectal cancer, esophageal cancer, myelodysplastic syndrome, multiple myeloma, gliomas, non-small cell lung cancer, stomach cancer, acute myeloid leukemia, hepatocellular carcinoma, breast cancer, ovarian cancer or prostate cancer.

13. The method of claim 1, wherein said inhibitor is a compound selected from the group consisting of azoles, aminoalkanimidazoles, aminoalkantriazoles, acylated aminoalkanimidazoles, and acylated aminoalkantriazoles.

14. The method of claim 1, wherein said inhibitor is selected from the group consisting of ketoconazole, clotrimazole, fluconazole, itraconazole, and liarozole.

15. The method of claim 1, wherein said azole compound has a bulky substituent attached at the carbon atom which is in the alpha position relative to the azole.

16. The method of claim 15, wherein said substituent is phenyl, naphthyl, thienyl, or pyridyl; or phenyl, naphthyl, thienyl or pyridyl monosubstituted by halogen, (C1-4) alkoxy, (C1-4)alkyl, di-(C1-4)alkylamino or cyano.

17. The method of claim 16, wherein said inhibitor is selected from the group consisting of (R)-SDZ-286907, (R)-SDZ-287871, (R)-VAB636, (R)-VID400, and (S)-SDZ-285428.

18. The method of claim 1, wherein said inhibitor is represented by Formula IV 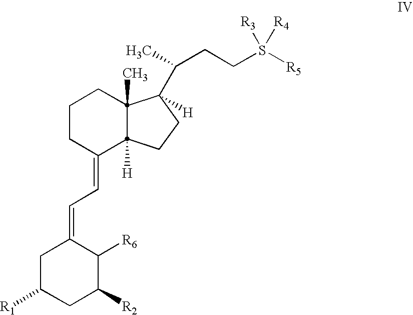
wherein R1 and R2 are each independently selected from the group consisting of hydrogen, OR′, —C(O)H, and —C(O)R′;
wherein R′ is selected from the group consisting of a C1 to C6 alkyl, a cycloalkyl, phenyl, an alkylaryl, an arylalkyl, and a heteroaryl; each of which can be optionally substituted with at least one halogen, thiol, mercapto, hydroxyl, or amino group;
wherein R3, R4 and R5 are each independently selected from the group consisting of hydrogen, hydroxyl, oxy, imine, phenyl, a C1 to C6 alkyl, alkenyl, cycloalkyl or cycloalkenyl, an alkylaryl, an arylalkyl, and a heteroaryl; each of which can be optionally substituted with at least one halogen, thiol, mercapto, hydroxyl, or amino group; and
wherein R6 is hydrogen, ═CH2 or a C1 to C6 alkyl, alkenyl, cycloalkyl, or cycloalkenyl, each of which can be optionally substituted with at least one halogen, thiol, mercapto, hydroxyl, or amino group;
or a pharmaceutically acceptable salt, hydrate, solvate, ester, or isomer thereof.

19. The method of claim 18, wherein said inhibitor is represented by Formula V 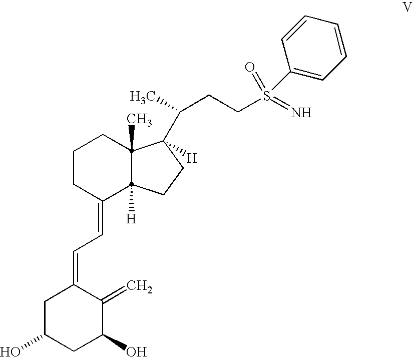
or a pharmaceutically acceptable salt, solvate, hydrate, ester or isomer thereof.

20. The method of claim 18, wherein said inhibitor is represented by Formula VI 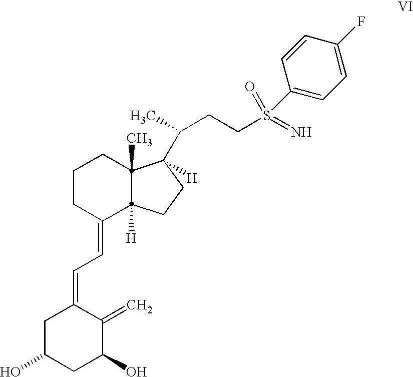
or a pharmaceutically acceptable salt, solvate, hydrate, ester or isomer thereof.

21. The method of claim 18, wherein said inhibitor is selected from the group consisting of compounds IIa, IIb, IIc, IId, IIe, IIf, IIg, IIh, IIi, IIj, and IIk as shown in FIG. 2 and pharmaceutically acceptable salts, solvates, hydrates, esters, or isomers thereof.

22. The method of claim 1 wherein the cancer is colorectal cancer and the cancer therapeutic is selected from the group consisting of 5-fluorouracil, capecitabine, tegafur with uracil, oxaliplatin, and irinotecan

23. The method of claim 1 wherein the cancer is esophageal cancer and the cancer therapeutic is selected from the group consisting of cisplatin and 5-fluorouracil.

24. The method of claim 1 wherein the cancer is MDS and the cancer therapeutic is selected from the group consisting of thalidomide, lenalidomide, a receptor tyrosine kinase inhibitor, SU5146, arsenic trioxide, bevacizumab, a farnesyltransferase inhibitor, tipifarnib, and lonafamib.

25. The method of claim 1 wherein the cancer is multiple myeloma and the cancer therapeutic is selected from the group consisting of melphalan, doxorubicin, cyclophosphamide, vincristine, and thalidomide.

26. The method of claim 1 wherein the cancer is glioma and the cancer therapeutic is selected from the group consisting of temozolomide and methotrexate.

27. The method of claim 1 wherein the cancer is non-small cell lung cancer and the cancer therapeutic is selected from the group consisting of cisplatin and cyclophosphamide.

28. The method of claim 1 wherein the cancer is stomach cancer and the cancer therapeutic is selected from the group consisting of epirubicin, cisplatin, and 5-fluorouracil, doxorubicin, and methotrexate, epirubicin, and methotrexate.

29. The method of claim 1 wherein the cancer is AML and the cancer therapeutic is selected from the group consisting of cytarabine, daunorubicin or idarubicin, 6-thioguanine, filgrastin, sargramostim.

30. The method of claim 1 wherein the cancer is AML and the cancer therapeutic is selected from the group consisting of doxorubicin, 5-fluorouracil, and gemcitabin.

31. The method of claim 1 wherein the cancer is breast cancer and the cancer therapeutic is selected from the group consisting of tamoxifen, raloxifene, fulvestrant, goserelin, LHRH, aromatase inhibitors, anastozole, letrozole, exemestane, doxorubicin, cyclophosphamide, paclitaxel, docetaxel and gemcitabine, and trastuzumab.

32. The method of claim 1 wherein the cancer is ovarian cancer and the cancer therapeutic is selected from the group consisting of cisplatin, carboplatin, paclitaxel, doxorubicin, altretamine, and topotecan hydrochloride.

33. The method of claim 1 wherein the cancer is prostate cancer and the cancer therapeutic is selected from the group consisting of docetaxel, mitoxantrone hydrochloride, estramustine sodium phosphate, an LHRH agonist, an estrogen, and an anti-androgen.
